(19) United States
(12) Patent Application Publication
(10) Pub. No.: US 2025/0311690 A1
(43) Pub. Date: Oct. 9, 2025

(54) SWISS CHARD VARIETY 'RHDF-611 23'

(71) Applicant: Pinnacle Seed, Inc., Carmel, CA (US)

(72) Inventors: Mike VANOLI, Carmel, CA (US); Christopher H. BECKER, Mount Vernon, WA (US)

(73) Assignee: Pinnacle Seed, Inc., Carmel, CA (US)

(21) Appl. No.: 19/173,408

(22) Filed: Apr. 8, 2025

Related U.S. Application Data
(60) Provisional application No. 63/631,366, filed on Apr. 8, 2024.

(51) Int. Cl. A01H 6/02; A01H 5/12

(52) U.S. Cl. CPC A01H 6/024; A01H 5/12

(57) ABSTRACT

New Swiss chard variety designated ‘RHdf-611 23’ exhibits stability and uniformity.

### CROSS-REFERENCE TO RELATED APPLICATION

This application claims the benefit of U.S. Provisional Application No. 63/631,366, filed Apr. 8, 2024, which is hereby incorporated by reference in its entirety.

### FIELD

This invention relates to the field of plant breeding. In particular, this invention relates to a new Swiss chard, Beta vulgaris L., variety, ‘RHdf-611 23’.

### BACKGROUND

Swiss chard (Beta vulgaris L.) is a green, leafy vegetable grown for its edible leaves and petioles. It has excellent nutritional value, providing a high amount of vitamins A, C, and K, as well as minerals such as iron, magnesium, and potassium. As such, Swiss chard often serves a similar role to spinach in salads, stews, and other dishes. In addition to agricultural uses, Swiss chard can serve as a decorative garden vegetable. Its attractive, vibrantly colored petioles provide Swiss chard with ornamental value.

Due to Swiss chard's growth close to the ground and low petiole and leaf attitude, the plant is typically harvested by the leaf as opposed to the entire plant. This results in a harvesting process that is inefficient and time consuming. Thus, there is a need for an improved Swiss chard variety with a very erect plant type to enable harvesting of the whole plant in a single pass.

### SUMMARY

In order to meet these needs, the present invention is directed to an improved Swiss chard variety with very erect leaves and petioles, thus generating a very erect plant type, as well as a dwarf plant size. This enables harvesting of the whole plant in a single pass.

In one embodiment, the present invention is directed to Swiss chard, Beta vulgaris L., seed designated as ‘RHdf-611 23’ having ATCC Accession Number X1. In one embodiment, the present invention is directed to a Beta vulgaris L. Swiss chard plant and parts isolated therefrom produced by growing ‘RHdf-611 23’ Swiss chard seed. In another embodiment, the present invention is directed to a Beta vulgaris L. plant and parts isolated therefrom having all the physiological and morphological characteristics of a Beta vulgaris L. plant produced by growing ‘RHdf-611 23’ Swiss chard seed having ATCC Accession Number X1. In still another embodiment, the present invention is directed to an F₁ hybrid Beta vulgaris L. Swiss chard seed, plants grown from the seed, and a plant isolated therefrom having ‘RHdf-611 23’ as a parent, where ‘RHdf-611 23’ is grown from ‘RHdf-611 23’ Swiss chard seed having ATCC Accession Number X1.

Swiss chard plant parts include, without limitation, a microspore, pollen, an ovary, an ovule, a cutting, a root, a stem, a cell, a protoplast, a leaf, a petiole, a cotyledon, a hypocotyl, a meristematic cell, a root, a root tip, an anther, a flower, a seed, or a stem. In another embodiment, the present invention is further directed to microspores, pollen, ovaries, ovules, cuttings, stems, cells, protoplasts, leaves, cotyledons, hypocotyls, meristematic cells, roots, root tips, anthers, flowers, or seeds isolated from ‘RHdf-611 23’ Swiss chard plants. In another embodiment, the present invention is further directed to tissue or cell culture of ‘RHdf-611 23’ Swiss chard plants, and to Swiss chard plants regenerated from the tissue or cell culture, where the plant has all of the morphological and physiological characteristics of ‘RHdf-611 23’ Swiss chard plants. In another embodiment, the present invention is further directed to tissue cell culture produced from protoplasts or cells of a Swiss chard plant, wherein the Swiss chard plant is produced by growing ‘RHdf-611 23’ Swiss chard seed having ATCC Accession Number X1.

In another embodiment, the present invention relates to a method of making Beta vulgaris L. seeds, including crossing the Swiss chard plant of any of the preceding embodiments with another Swiss chard plant and harvesting seed therefrom. In some embodiments, the method includes planting the seed to produce F1 progeny. In some embodiments, the method further includes (a) selecting one or more of the F1 progeny for plants that have a desired trait to produce selected progeny plants; (b) crossing the selected progeny plants with the Swiss chard plant of any of the preceding embodiments to produce backcross progeny plants; (c) selecting for backcross progeny plants that have the desired trait and all of the physiological and morphological characteristics of the Swiss chard plant of any of the preceding embodiments when grown in the same environmental conditions to produce selected backcross progeny plants; and (d) repeating steps (b) and (c) three or more times in succession to produce selected fourth or higher backcross progeny plants that comprise the desired trait and all of the physiological and morphological characteristics of the Swiss chard plant of any of the preceding embodiments when grown in the same environmental conditions.

In another aspect, the present invention relates to a method of producing a seed of a Swiss chard plant derived from the Swiss chard plant of any of the preceding embodiments, including crossing a Beta vulgaris L. plant of any of the preceding embodiments with a second Swiss chard plant, whereby seed of a derived Swiss chard plant forms. In another embodiment, the present invention relates to a derived Swiss chard plant produced by growing the seed of any of the preceding embodiments. In some embodiments, the method further includes (a) crossing a plant grown from Beta vulgaris L. seed derived from the Swiss chard plant of any of the preceding embodiments with itself or with a second Swiss chard plant to yield additional seed of a derived Swiss chard plant; (b) growing the additional derived Swiss chard plants; and (c) repeating the crossing and growing of steps (a) and (b) for an additional 3-10 generations to generate further Swiss chard plants derived from the Swiss chard plant of any of the preceding embodiments.

In another embodiment, the present invention relates to a method of producing a Swiss chard petiole from the plant of any of the preceding embodiments, said method including growing the plant of any of the preceding embodiments until it produces at least one petiole; and harvesting the petiole.

In another aspect, the present invention relates to a method of producing market-ready plant material, said method comprising obtaining the plant of any of the preceding embodiments and producing said market-ready plant material therefrom. In some embodiments, the market-ready plant material is a whole plant of Swiss chard variety ‘RHdf-611 23’.

### BRIEF DESCRIPTION OF THE DRAWINGS

The patent or application file contains at least one drawing executed in color. Copies of this patent or patent application publication with color drawings will be provided by the office upon request and payment of the necessary fee.

FIG. 1 shows whole plants of Swiss chard variety ‘RHdf-611 23’ in the field.

FIG. 2 shows whole plants, petioles, lower surfaces of leaves, and roots of Swiss chard variety ‘RHdf-611 23’.

FIG. 3 shows a comparison of Swiss chard variety ‘RHdf-611 23’ (left) to Swiss chard variety ‘Bright Lights’ (unpatented, right). The image demonstrates the absent/very weak undulation of margin on the leaves in Swiss chard variety ‘RHdf-611 23’, compared to wavy or curled leaves of ‘Bright Lights’ (unpatented).

FIG. 4 shows another comparison of Swiss chard variety ‘RHdf-611 23’ (bottom) to ‘Bright Lights’ (unpatented, top). The image demonstrates the dwarf plant size of Swiss chard variety ‘RHdf-611 23’, compared to the plant size of ‘Bright Lights’ (unpatented).

### DETAILED DESCRIPTION

### Definitions

In order to more clearly understand the invention, the following definitions are provided:

Attitude of Leaf Blade: Attitude of leaf blades was determined by visual inspection and subsequently confirmed with marker analysis.

Maturity Date: Maturity refers to the stage when the plants are of full size or optimum weight, in marketable form or shape to be of commercial or economic value.

Plant Measurements: All plant measurements were performed via visual inspection.

Petiole Coloration: Petiole color was determined by visual inspection.

Taking into account these definitions, the present invention is directed to seeds of the Swiss chard variety ‘RHdf-611 23’, plants produced by growing ‘RHdf-611 23’ Swiss chard seeds, leaves and petioles isolated or harvested from the plants, one or more plants selected from a collection of ‘RHdf-611 23’ plants and seeds derived or produced therefrom; plants produced by crossing a Swiss chard plant with a ‘RHdf-611 23’ Swiss chard plant and seeds derived or produced therefrom.

### Objective Description of the Variety ‘RHdf-611 23’

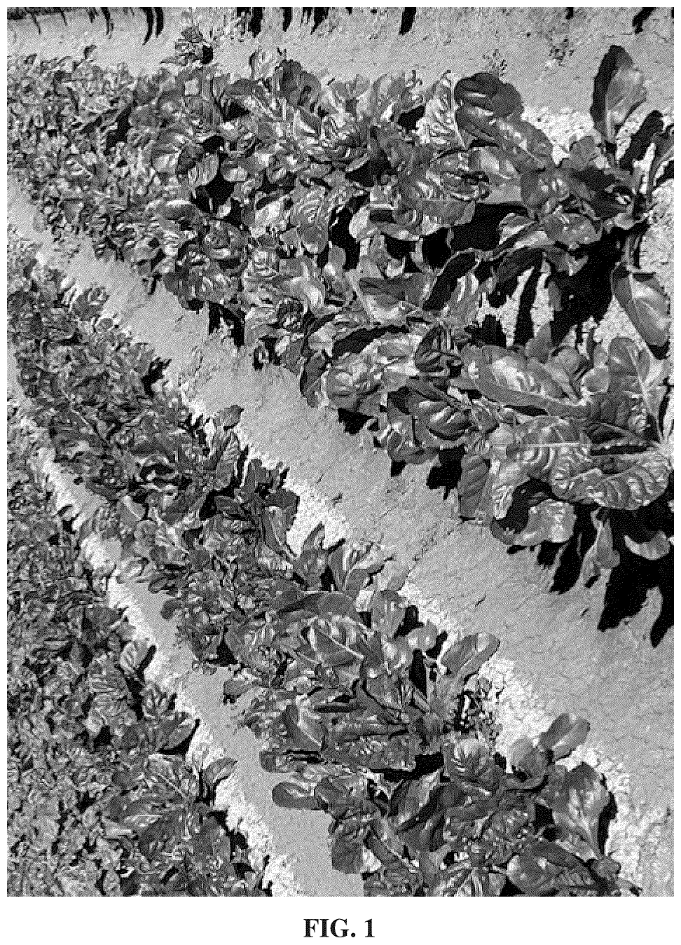

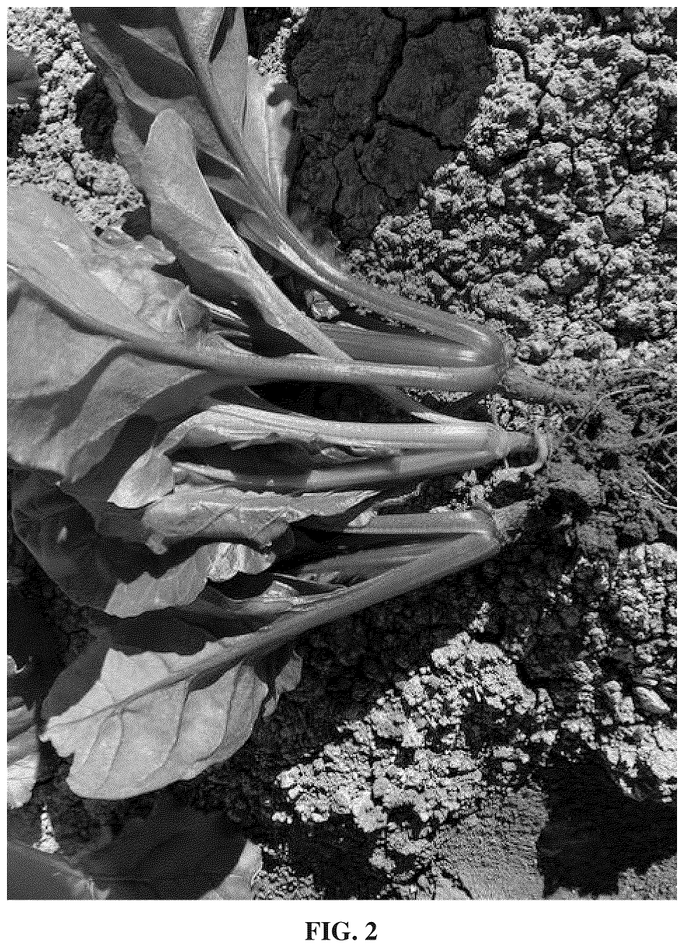

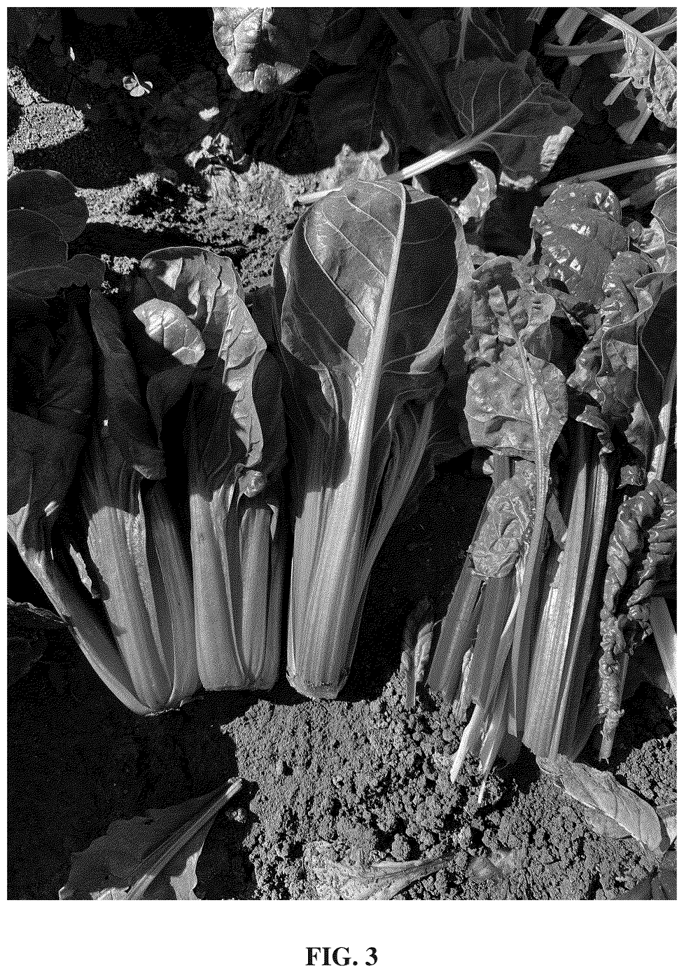

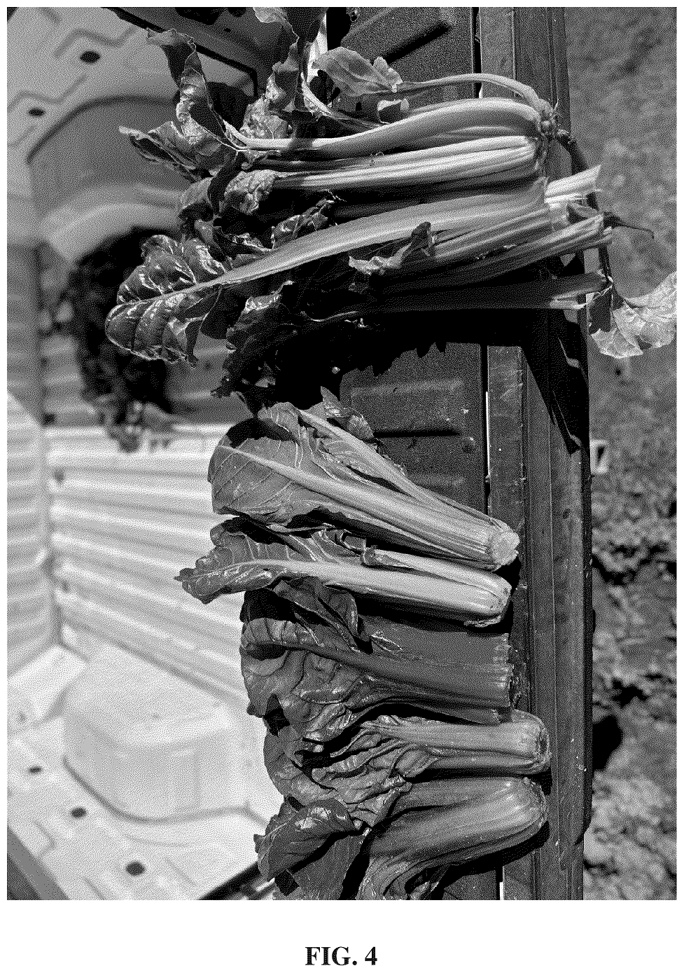

‘RHdf-611 23’ is a Swiss chard variety with a very erect leaf and petiole attitude, dwarf plant size, and multicolored petioles, including red, yellow, orange, pink, and white. In particular, Swiss chard variety ‘RHdf-611 23’ produces dwarf-type plants similar to bok choy (Brassica rapa subsp. chinensis). This variety is distinct and unique to all other Swiss chard varieties due to its very erect leaf and petiole attitude, which aids in the ease of harvest. This enables the whole plant to be harvested in a single pass as opposed to individual leaves/stems in traditional Swiss chard. Additionally, ‘RHdf-611 23’ produces leaves with a broad elliptic shape, absent/very weak undulation of the margin, and weak blistering. FIG. 1 depicts whole plants of Swiss chard variety ‘RHdf-611 23’ in the field, and FIG. 2 depicts whole plants, petioles, lower surfaces of leaves, and roots of Swiss chard variety ‘RHdf-611 23’. FIGS. 3 and 4 show comparisons of Swiss chard variety ‘RHdf-611 23’ to Swiss chard variety ‘Bright Lights’ (unpatented).

The variety has shown uniformity and stability for the traits, within the limits of environmental influence for the traits. It has been self-pollinated a sufficient number of generations with careful attention to uniformity of plant type. The line has been increased with continued observation for uniformity. No variant traits have been observed or are expected in variety ‘RHdf-611 23’.

Swiss chard variety ‘RHdf-611 23’ has the following morphologic and other characteristics:

### Germity:

Multigerm

### Seedling:

Coloration of Hypocotyl: Multicolored; Individual plants may be red, yellow, orange, or white.

### Leaf:

Leaf: Attitude of Petiole: Erect
Leaf: Attitude of Blade: Erect
Leaf Length (Including Petiole): Short
Leaf Blade Length (cm): 30.5
Leaf Blade Width (cm): 20.5
Leaf Blade Shape: Broad elliptic
Leaf Blade Color: Mainly green
   Intensity of Green Color: Medium
Leaf Blade: Coloration of Veins: Strong red color, strong orange color, strong yellow color, or strong white color
Leaf Blade: Undulation of Margin: Absent or very weak
Leaf Blade: Blistering: Weak

### Petiole:

Petiole: Width of Base (at Root Insertion): Medium
Petiole: Main Color of Lower Side: Red, orange, yellow, or white

### Root:

Root Position in Soil: Medium
Root Length (cm): 28.5
Root Width (cm): 4.5
Root Ratio (Length/Width Ratio): 6.3
Root Corkiness: Absent or very weak
Root External Color: White or red
Root: Main Color of Flesh: White
Root: Intensity of Main Color of Flesh: Medium
Root: Prominence of Rings: Absent or very weak

### Bolting:

Bolting Tendency (from an Early Sowing): Absent or weak

### Comparisons to Commercial Swiss Chard Varieties

Table 1 below compares characteristics of Swiss chard variety ‘RHdf-611 23’ with the Swiss chard varieties ‘Bright Yellow’ (PVP Certificate No. 200000052), ‘Prima Rossa’ (PVP Certificate No. 200900006), and ‘Peppermint’ (PVP Certificate No. 201100151). Column 1 lists the characteristics, column 2 shows the characteristics for Swiss chard variety ‘RHdf-611 23’, column 3 shows the characteristics for Swiss chard variety ‘Bright Yellow’, column 4 shows the characteristics for Swiss chard variety ‘Prima Rossa’, and column 5 shows the characteristics for Swiss chard variety ‘Peppermint’.

TABLE 1

| | | Bright | Prima | |
|---|---|---|---|---|
| Characteristic | RHdf-611 23 | Yellow | Rossa | Peppermint |
| Leaf | Very Erect | Semi-erect | Erect | Erect |
| Attitude/ | | | | |
| Carriage | | | | |
| Leaf Blade | 30.5* | 37 | 10 | 25 |
| Length (cm) | | | | |
| Leaf Blade | 20.5* | 19 | 5 | 15 |
| Width (cm) | | | | |
| Leaf Color | Medium | Dark | Dark | Medium |
| | Green | Green | Red | Green |
| Leaf | Absent or Very | Wavy/Curled | Wavy | Wavy |
| Undulation | Weak^{†} | | | |
| of Margin | | | | |
| Leaf Blade | Weak | Blistered | Blistered | Blistered |
| Surface | Blistering | | | |
| Petiole Main | Red, Orange, | Yellow | Red | White |
| Color | or Yellow | | | |
| Vein Color | Strong Red, | Yellow | Green | White |
| | Orange, or | | or Red | |
| | Yellow | | | |
| Leaf Blade | Weak | Blistered | Blistered | Blistered |
| Blistering | | | | |

*measurement includes petiole;
^{†}determined on a scale of absent to very strong undulation

FIG. 3 shows a comparison of Swiss chard variety ‘RHdf-611 23’ (left) to traditional colored chard (right). The image demonstrates the absent/very weak undulation of margin on the leaves in Swiss chard variety ‘RHdf-611 23’, compared to wavy or curled leaves of traditional colored chard. Notably, Swiss chard variety ‘RHdf-611 23’ has been harvested as a whole plant, enabled by its very erect plant type and dwarf plant size. This is in contrast to the traditional colored chard, which has been harvested as individual petioles. FIG. 4 shows another comparison of Swiss chard variety ‘RHdf-611 23’ (bottom) to traditional colored chard (top). The image shows full plants of ‘RHdf-611 23’ and full plants and petioles of the traditional colored chard, and demonstrates the dwarf plant size of Swiss chard variety ‘RHdf-611 23’ compared to the plant size of traditional colored chard.

### Further Embodiments

### Breeding

In Swiss chard breeding, lines are selected for their appropriate characteristics. For example, one line may be selected for its size and the color of its leaves. Another line may be selected for attitude of leaves. Crosses are made, for example, to produce erect-leaf and petiole varieties with medium green leaves and dwarf size.

### Selection

In addition to crossing, selection may be used to identify and isolate new Swiss chard lines. In Swiss chard selection, Swiss chard seeds are planted, the plants are grown, and single plant selections are made of plants with desired characteristics. Such characteristics may include improved plant and leaf size, deeper or darker green leaf color, leaf or petiole attitude, etc. Seed from the single plant selections are harvested, separated from seeds of the other plants in the field and re-planted. The plants from the selected seed are monitored to determine if they exhibit the desired characteristics of the originally selected line. Selection work is continued over multiple generations to increase the uniformity of the new line.

### Growth Conditions

Swiss chard prefers cool growth temperatures, but it tolerates heat well and rarely bolts. Swiss chard does best in full sun, although it tolerates partial shade and likes fertile, well-worked soil with good drainage and high organic content. Swiss chard grows well regardless of soil type, daylength or temperature. Swiss chard is quite cold tolerant and will continue to grow in the garden through frosts until temperatures drop to about 25° F. Swiss chard may be sown after soils reach 40° F. Seeds germinate best at 55-75° F. and require 7-14 days to emerge. Temperatures above 80° F. reduce seed germination. Seeds may be planted ½ inch deep. Stands are thinned when plants have 3-4 true leaves.

### Containers and Packaging of Leaves

Swiss chard leaves are sold in packaged form, including without limitation as pre-packaged Swiss chard salad or as frozen Swiss chard. Examples of suitable Swiss chard packaging may be found in U.S. Pat. No. 5,523,136. Such suitable packaging includes, without limitation, packaging film, and packages from such packaging film, including such packaging containing leafy produce, and methods for making and using such packaging film and packages, which are suitable for use with the Swiss chard leaves of the invention.

### DEPOSIT INFORMATION

### Swiss Chard Variety ‘RHdf-611 23’

A deposit of at least 625 seeds of the Swiss chard variety ‘RHdf-611 23’ was made with the American Type Culture Collection (ATCC), ATCC Patent Depository, 10801 University Boulevard, Manassas, Virginia, 20110, USA, and assigned ATCC number X1. The seeds deposited with the ATCC on DATE were obtained from the seed of the variety maintained by Pinnacle Seed, Inc., P.O. Box 222672, Carmel, California 93923, United States of America since prior to the filing date of the application. Access to this deposit will be available during the pendency of this application to persons determined by the Commissioner of Patents and Trademarks to be entitled thereto under 37 C.F.R. § 1.14 and 35 U.S.C. § 122. Upon issuance, the Applicant will make the deposit available to the public consistent with all of the requirements of 37 C.F.R. § 1.801-1.809. This deposit of the Swiss chard variety ‘RHdf-611 23’ will be maintained in the ATCC, which is a public depository, for a period of 30 years, or at least 5 years after the most recent request for a sample of the deposit, or for the effective life of the patent, whichever is longer, and will be replaced if it becomes nonviable during that period. Applicant has no authority to waive any restrictions imposed by law on the transfer of biological material or its transportation in commerce. Applicant does not waive any infringement of rights granted under this patent or under the Plant Variety Protection Act (7 USC 2321 et seq.).

What is claimed:

1. A Beta vulgaris L. seed designated as ‘RHdf-611 23’, representative sample of seed having been deposited under ATCC Accession Number X1.

2. A Beta vulgaris L. plant produced by growing the seed of claim 1.

3. A plant part from the plant of claim 2, wherein said part is a leaf, a petiole, or a portion thereof.

4. The plant part of claim 3, wherein said part is a petiole.

5. A Beta vulgaris L. plant having all the physiological and morphological characteristics of the Beta vulgaris L. plant of claim 2.

6. A plant part from the plant of claim 5, wherein said part is a leaf, a petiole, or a portion thereof.

7. The plant part of claim 6, wherein said part is a petiole.

8. An F₁ hybrid Beta vulgaris L. plant having ‘RHdf-611 23’ as a parent wherein ‘RHdf-611 23’ is grown from the seed of claim 1.

9. A pollen grain or an ovule of the plant of claim 2.

10. A tissue or cell culture produced from protoplasts or cells from the plant of claim 2, wherein said cells or protoplasts are produced from a plant part selected from the group consisting of root, root tip, meristematic cell, stem, hypocotyl, petiole, cotyledon, leaf, flower, anther, pollen, pistil, embryo, seed, and fruit.

11. A Beta vulgaris L. plant regenerated from the tissue or cell culture of claim 10, wherein the plant has all of the morphological and physiological characteristics of a Beta vulgaris L. plant produced by growing seed designated as ‘RHdf-611 23’ having ATCC Accession Number X1.

12. A method of making Beta vulgaris L. seeds, said method comprising crossing the plant of claim 2 with another Swiss chard plant and harvesting seed therefrom.

13. The method of claim 12, wherein the method further comprises planting the harvested seed to grow F₁ progeny.

14. A method of producing a seed of a ‘RHdf-611 23’-derived Swiss chard plant derived from the plant of claim 2, comprising crossing the plant of claim 2 with a second Swiss chard plant, whereby seed of a Swiss chard plant derived from the plant of claim 2 forms.

15. A ‘RHdf-611 23’-derived Swiss chard plant produced by growing the seed of claim 14.

16. The method of claim 15, further comprising:
(a) crossing the ‘RHdf-611 23’-derived Swiss chard plant with itself or a second Swiss chard plant to yield additional seed of the ‘RHdf-611 23’-derived Swiss chard plant;
(b) growing the additional seed of the ‘RHdf-611 23’-derived Swiss chard plant of step (a) to yield additional ‘RHdf-611 23’-derived Swiss chard plants; and
(c) repeating the crossing and growing steps of (a) and (b) for an additional 3-10 generations to generate further ‘RHdf-611 23’-derived Swiss chard plants.

17. A method of producing a Swiss chard petiole, said method comprising:
(a) growing the plant of claim 2 until it produces at least one petiole; and
(b) harvesting the petiole.

18. A method of producing market-ready plant material, said method comprising obtaining the plant of claim 2 and producing said market-ready plant material therefrom.

19. The method of claim 18, wherein the market-ready plant material is a whole plant of Swiss chard variety ‘RHdf-611 23’.
